(19) United States
(12) Patent Application Publication
(10) Pub. No.: US 2026/0272808 A1
(43) Pub. Date: Sep. 17, 2026

(54) PREPARATION METHOD AND USE OF COMPOSITION OF TRIPLE-HELIX COLLAGEN AND ANTARCTIC GLYCOPROTEIN

(71) Applicant: Hangzhou Yinian Brand Management Co., Ltd., Qianmo (CN)

(72) Inventors: Ke ZHENG, Hangzhou (CN); Disi Zhu, Hangzhou (CN); Hengshan Ma, Hangzhou (CN); Xin Gu, Hangzhou (CN); Jinbao Huang, Hangzhou (CN)

(21) Appl. No.: 19/366,608

(22) Filed: Oct. 23, 2025

(30) Foreign Application Priority Data
Jan. 16, 2025 (CN) 202510066400.0

(51) Int. Cl. A61K 8/65; A61K 8/64; A61Q 19/08; C07K 1/34; C07K 14/435; C07K 14/78; C12P 21/00; C12P 21/06

(52) U.S. Cl. CPC A61K 8/65; A61K 8/64; A61Q 19/08; C07K 1/34; C07K 14/43509; C07K 14/78; C12P 21/005; C12P 21/06

(57) ABSTRACT

The present application discloses a preparation method and use of a composition of triple-helix collagen and Antarctic glycoprotein. A method for preparing a composition of triple-helix collagen and Antarctic glycoprotein provided by the present application includes: mixing MaAFP antifreeze protein, Antarctic krill glycoprotein and triple-helix collagen. The composition of triple-helix collagen and Antarctic glycoprotein prepared by the method of the present application has a protective effect on UVB induced Hacat cells, can effectively inhibit inflammatory cytokines and cellular oxidative stress level, and has good use prospects in the preparation of anti-aging products.

### CROSS-REFERENCE TO RELATED APPLICATION

The present application claims the priority and benefit of Chinese patent application No. 202510066400.0, filed on Jan. 16, 2025. The entirety of Chinese patent application No. 202510066400.0 is hereby incorporated by reference herein and made a part of this specification.

### REFERENCE TO AN ELECTRONIC SEQUENCE LISTING

The contents of the electronic sequence listing (SequenceListing.xml; Size: 2,064 bytes; and Date of Creation: Oct. 22, 2025) is herein incorporated by reference.

### TECHNICAL FIELD

The present application belongs to the field of biotechnology and beauty and, in particular, relates to a preparation method and use of a composition of triple-helix collagen and Antarctic glycoprotein.

### BACKGROUND ART

Skin aging is an inevitable stage in the process of biological life activities. It refers to a progressive, genetically influenced, and complex process of irreversible decline, throughout the body, in morphological structure and physiological function, that occurs with increasing age after an organism reaches maturity. Skin aging refers to the process of skin deterioration in the external shape, structure and function of skin under the influence of exogenous or endogenous factors. Endogenous aging refers to the natural aging of the human body, and exogenous aging mainly refers to photoaging. The endogenous aging and the exogenous aging of the skin are interconnected and affect each other. The clinical manifestations of skin aging include dryness, roughness, thinning, reduced elasticity, dullness, slackness and atrophy of skin, along with varying degrees of wrinkles, pigmentation, telangiectasia, and the physiological structure disorder and functional degradation of skin. Therefore, how to alleviate the aging process of skin has become a hot topic in the field of anti-aging research.

The triple-helix structure of collagen is a right-handed a right-handed superhelical rod-like structure formed by three independent and left-handed helical α-peptide chains which are interconnected and intertwined, around the same central axis, by hydrogen bonds formed therebetween. This structure is quite stable, and glycine, proline and hydroxyproline play an important role in maintaining its structural stability. In the structure, there is one glycine in every two amino acids. The bends formed by glycines fill the interior of the helices, and proline and hydroxyproline chains form smooth bends around the helices, thus forming a tight and stable three-dimensional helix structure. In the skin, collagen, as a main component of the true cortex, helps maintain the elasticity and water retention of the skin. As we get older, the content of collagen gradually decreases, causing the skin to lose elasticity and luster, get wrinkled and become slack. As an active polymer material, collagen is very easy to degrade and has a short shelf life at room temperature and high temperature. Therefore, how to maximize the activity of collagen in an elevated temperature environment has become a research hotspot in the fields of biotechnology and aging resistance.

In the extremely cold Antarctic glacier land, various prokaryotes inhabit barren soil, rocky terrain, and countless lakes. This extreme low-temperature environment will not become an obstacle to the survival of microbial communities. Psychrophiles and psychrotrophs are two types of extremophiles that thrive in cold environments. Similarly, in the extremely cold and oxygen-rich Antarctic seas, Antarctic krill have become a specialized species in the unique Antarctic ecosystem. Antifreeze proteins (AFPs) and antifreeze glycoproteins (AFGP), collectively called Antarctic glycoprotein, are one of the most important characteristics of extremophiles and Antarctic fish and shrimp adapting to extreme cold environments. Antarctic glycoprotein was found in extracellular products of Pseudoalteromonas and Antarctic krill extracts. Antarctic glycoprotein, also known as ice binding proteins (IBPs), can bind to ice crystals or low-temperature sensitive substances such as cell membranes, enzymes, etc. at low temperatures to inhibit the formation and growth process of ice crystals, while stabilizing low-temperature sensitive substances. The activity of Antarctic glycoprotein to inhibit the growth of ice crystals is called recrystallization inhibition (RI) activity. As a rare component that can protect skin in severe cold environments, Antarctic glycoprotein can prevent freezing and dryness, and protect skin from injury. In addition, Antarctic glycoprotein can lock moisture on the surface of the skin and absorb moisture in the air. Moreover, Antarctic glycoprotein can increase the amount of collagen and elastin in the skin, stimulate the healthy growth of the skin from the inside out, and has the effect of promoting wound healing. Besides, Antarctic glycoprotein can stimulate cell vitality and form a silky protective film on the surface of the skin, preventing and repairing skin injury caused by heavy loss of water, and making the skin tight, elastic and radiant.

Although the triple-helix collagen and the Antarctic glycoprotein have shown good use prospects in the technical field of aging resistance, the existing preparation processes for obtaining triple-helix collagen and Antarctic glycoprotein cannot meet practical use needs. Moreover, there is currently no research on combining triple-helix collagen with Antarctic glycoprotein to resist skin aging. Therefore, providing a method for preparing a composition of triple-helix collagen and Antarctic glycoprotein holds significant reference value for the research of anti-aging products.

### SUMMARY

In view of the above shortcomings, the present application provides a preparation method and use of a composition of triple-helix collagen and Antarctic glycoprotein. A method for preparing a composition of triple-helix collagen and Antarctic glycoprotein provided by the present application includes: mixing MaAFP antifreeze protein, Antarctic krill glycoprotein and triple-helix collagen. The composition of triple-helix collagen and Antarctic glycoprotein prepared by the method of the present application has a protective effect on UVB induced Hacat cells, can effectively inhibit inflammatory cytokines and cellular oxidative stress level, and has good use prospects in the preparation of anti-aging products.

The technical solutions of the present application include:

A method for preparing a composition of triple-helix collagen and Antarctic glycoprotein, including:
S1, adding a lyophilized Antarctic krill powder to distilled water to obtain a first mixture, adjusting a pH value of the first mixture, then adding an enzyme mixture to the first mixture for enzymatic hydrolysis, and then performing inactivation and collecting a supernatant;
S2, performing dialysis and concentration on the supernatant, and then adding ethanol, standing still overnight, collecting a precipitate, and lyophilizing the precipitate to obtain a crude Antarctic krill glycoprotein product;
S3, dissolving the crude Antarctic krill glycoprotein product in distilled water to obtain a crude Antarctic krill glycoprotein solution, adding a MaAFP antifreeze protein solution into the crude Antarctic krill glycoprotein solution to obtain a second mixture, using a cation exchange medium to adsorb proteins in the second mixture, and then performing elution and separation, and collecting an eluent which is an Antarctic glycoprotein solution; and
S4, mixing the Antarctic glycoprotein solution and a triple-helix collagen hydrogel solution thoroughly at a volume ratio of 0.4-1.5:1 to obtain a third mixture, and lyophilizing the third mixture to obtain the composition of triple-helix collagen and Antarctic glycoprotein.

Specifically, a method for preparing the lyophilized Antarctic krill powder used in the step S1 includes: washing Antarctic krill, and then performing lyophilizing, pulverizing and sieving to obtain a uniform lyophilized Antarctic krill powder.

Preferably, the sieving is performed using a 10-20 mesh sieve.

Specifically, a ratio of the lyophilized Antarctic krill powder to the distilled water in the step S1 is 1:(40-60) g/mL.

Preferably, a ratio of the lyophilized Antarctic krill powder to the distilled water in the step S1 is 1:50 g/mL.

Preferably, the pH value in the step S1 is 6.0 to 7.0.

Specifically, the enzyme mixture in the step S1 consists of a cellulase, papain and a neutral protease.

Preferably, a weight ratio of the cellulase to the papain to the neutral protease in the step S1 is 2:1.5:1.

Specifically, the enzymatic hydrolysis in the step S1 is performed at a temperature of 50° C. to 60° C.

Preferably, the enzymatic hydrolysis in the step S1 is performed at the temperature of 50° C. to 51° C., 51° C. to 52° C., 52° C. to 53° C., 53° C. to 54° C., or 54° C. to 55° C.

Specifically, a time of the enzymatic hydrolysis in the step S1 is 2 h to 4 h.

Preferably, the time of the enzymatic hydrolysis in the step S1 is 2 h to 3 h, or 3 h to 4 h.

Specifically, the inactivation in the step S1 is performed at a temperature of 90° C. to 100° C.

Preferably, the inactivation in the step S1 is performed at the temperature of 90° C. to 91° C., 91° C. to 92° C., 92° C. to 93° C., 93° C. to 94° C., 94° C. to 95° C., 95° C. to 96° C., 96° C. to 97° C., 97° C. to 98° C., 97° C. to 98° C., 98° C. to 99° C., or 99° C. to 100° C.

Specifically, a time of the inactivation in the step S1 is 15 min to 30 min.

Preferably, the time of the inactivation in the step S1 is 15 min to 16 min, 16 min to 17 min, 17 min to 18 min, 18 min to 19 min, 19 min to 20 min, 20 min to 21 min, 21 min to 22 min, 22 min to 23 min, 23 min to 24 min, or 24 min to 25 min.

Specifically, the collecting in the step S1 is realized by means of centrifugation.

Preferably, the centrifugation is performed at 3000-8000 rpm for 10-30 min.

Specifically, the dialysis in the step S2 includes the following steps: placing the supernatant into a dialysis bag, and performing dialysis using distilled water as a dialysis medium.

Preferably, a molecular cutoff of the dialysis bag is 10 kDa to 15 kDa, and may be 10 kDa, 11 kDa, 12 kDa, 13 kDa, 14 kDa or 15 kDa.

Preferably, the concentration in the step S2 is performed to concentrate a dialysate to ⅕ of an original volume.

Specifically, a final volume concentration of the ethanol in the step S2 is 70% to 80% v/v.

Preferably, the final volume concentration of the ethanol in the step S2 is 75% v/v.

Specifically, the collecting in step S2 is realized by means of centrifugation.

Preferably, the centrifugation is performed at 3000-8000 rpm for 10-30 min.

Specifically, an amino acid sequence of the MaAFP antifreeze protein in the step S3 is as shown in SEQ ID NO. 1.

SEQ ID NO. 1:
GSGNDQIVIDNDVEKWLDAGDGDNSIYVGDDVNRNNSAGIKTGSGDDDIF
VKDNVDSTIQTGGGNDRVQIGYDLGNGYHSAHINLGDGDNNLIIKNDVNN
YSTVHSGSGDDVVSIGDDVRYDADIQLGDGNDRLTIGDKIEKEVSINLGS
GNDVLVVGGKVSDEAWVDGGSGSDSVWFESYSRSDYNSDKDGIKSRFANF
ENFKFSDGTVIGNASAF.

Specifically, a method for preparing the MaAFP antifreeze protein solution in the step S3 includes:
\textcircled{1} inoculating pseudoalteromonas into a medium containing glutamic acid, proline, vitamin B1 and sucrose at an inoculation amount of 3% to 5%, and performing culture to obtain a bacterial solution;
\textcircled{2} performing centrifugation on the bacterial solution and then collecting a supernatant, filtering the supernatant, and collecting a filtrate;
\textcircled{3} ultrafiltering the filtrate and then performing dialysis, and collecting and lyophilizing a dialysis retentate to obtain a powder; and
\textcircled{4} dissolving the powder to obtain a mixture, using a cation exchange medium to adsorb proteins in the mixture and then performing elution and separation, and collecting an eluent to obtain the MaAFP antifreeze protein solution.

Preferably, the medium in the step S3\textcircled{1} is a medium containing the glutamic acid with a final concentration of 0.2 g/L to 0.6 g/L, the proline with a final concentration of 0.15 g/L to 0.45 g/L, the vitamin B1 with a final concentration of 15 mg/L to 30 mg/L, and the sucrose with a final concentration of 1 g/L to 3 g/L.

Preferably, the medium in the step S3\textcircled{1} is 2216E liquid medium.

Preferably, the pseudoalteromonas in the step S3\textcircled{1} is SHMCC JCM18779.

Preferably, the inoculation amount of the pseudoalteromonas in the step S3\textcircled{1} is 3% to 4%, or 4% to 5%.

Preferably, the culture in the step S3\textcircled{1} is performed at 15-20° C. and 200-500 r/min for 5-7 days.

Preferably, the centrifugation in the step S3\textcircled{2} is performed at 3000-8000 rpm for 10-15 min.

Preferably, the filtering in the step S3\textcircled{2} is performed using a 0.22 μm filter membrane.

Preferably, the ultrafiltering in the step S3\textcircled{3} is performed using a 15-30 kDa ultrafiltration membrane at a pressure of 5-8 bar.

Preferably, the dialysis in the step S3\textcircled{3} includes the following steps: placing the ultrafiltrate into a dialysis bag, and performing dialysis using PBS with a pH value of 7.4 as a dialysis medium.

Further preferably, a molecular cutoff of the dialysis bag used for dialysis is 10 kDa to 15 kDa, and may be 10 kDa, 11 kDa, 12 kDa, 13 kDa, 14 kDa or 15 kDa.

Further preferably, the dialysis in the step S3\textcircled{3} is performed at 20-25° C. for 24-48 h.

Preferably, a solution used for the dissolving in the step S3\textcircled{4} is PBS with a pH value of 7.4.

Specifically, the cation exchange medium in the step S3\textcircled{4} is at least one selected from a group consisting of: DEAE(N,N-diethylaminoethyl)Sepharose Fast Flow, Q(N,N-diethylamino-2-hydroxypropyl), SP(sulfopropyl)-Sephadex, and Sephadex G-100; and the Sephadex G-100 is further preferred.

Preferably, a solution used for elution in the step S3\textcircled{4} is PBS with a pH value of 7.4.

Specifically, the cation exchange medium in the step S3 is at least one selected from a group consisting of: DEAE(N,N-diethylaminoethyl)Sepharose Fast Flow, Q(N,N-diethylamino-2-hydroxypropyl), and SP(sulfopropyl)-Sephadex;
preferably, the cation exchange medium in the step S3 is the SP(sulfopropyl)-Sephadex.

Preferably, a solution used for the elution in the step S3 is a 10 mM Tris-HCl aqueous solution containing 0.5 wt %-0.9 wt % of a sodium salt.

Specifically, a volume ratio of the MaAFP antifreeze protein solution to the crude Antarctic krill glycoprotein solution in the step S3 is (0.3-1.0):1.

More specifically, the volume ratio of the MaAFP antifreeze protein solution to the crude Antarctic krill glycoprotein solution in the step S3 is (0.3-0.4):1, (0.4-0.5):1, (0.5-0.6):1, (0.6-0.7):1, (0.7-0.8):1, (0.8-0.9):1, or (0.9-1.0):1.

Preferably, the volume ratio of the MaAFP antifreeze protein solution to the crude Antarctic krill glycoprotein solution in step S3 is (0.4-0.5):1, (0.5-0.6):1, or (0.6-0.7):1.

Further specifically, the Antarctic glycoprotein solution and the triple-helix collagen hydrogel solution in the step S4 are mixed thoroughly at a volume ratio of (0.4-0.5):1, (0.5-0.6):1, (0.6-0.7):1, (0.7-0.8):1, (0.8-0.9):1, (0.9-1.0):1, (1.0-1.1):1, (1.1-1.2):1, (1.2-1.3):1, (1.3-1.4):1, or (1.4-1.5):1.

Preferably, the Antarctic glycoprotein solution and the triple-helix collagen hydrogel solution in the step S4 are mixed thoroughly at a volume ratio of (1.2-1.3):1.

Specifically, a method for preparing the triple-helix collagen hydrogel solution in the step S4 includes:
(1) soaking pig skin in an EDTA-containing trypsin solution, and then rinsing; then, soaking the pig skin in a detergent solution and shaking, and then rinsing; then, soaking the pig skin in a neutral protease solution, and then rinsing; and then soaking the pig skin in detergent solution and shaking, and then rinsing; and repeating the above steps for 1-3 times to obtain non-immunogenic pig skin;
(2) soaking the non-immunogenic pig skin in an α-1,3-galactosidase solution, and then rinsing to obtain precipitate A; adding a filter aid solution to the precipitate A, then stirring, filtering, and collecting a supernatant; concentrating the supernatant, then using a cation exchange medium to adsorb collagen in the supernatant, and then performing elution and separation to finally obtain an eluent;
(3) adding a modified polysaccharide solution with a concentration of 10 mg/mL to 40 mg/mL to the eluent from the step (2) for modification to obtain a hydrogel, and a mass ratio of the eluent to the modified polysaccharide solution is 5:1-1:3; and
(4) performing filtration and sterilization on the hydrogel obtained in step (3) with a 0.22 μm filter membrane to obtain the triple-helix collagen hydrogel solution.

More specifically, the detergent solution in the step S4 (1) is a sodium phosphate buffer with a pH value of 6.0-8.0, containing 0.05%-0.15 wt % of an ionic detergent; and the ionic detergent is a mixture of sodium dodecyl sulfate and Triton X-200 (p-Octylphenoxyethoxyethyl sodium sulfonate) at a mass ratio of 4:6;
preferably, the sodium phosphate buffer is a sodium phosphate buffer solution with a pH value of 7.2.

More specifically, in the EDTA-containing trypsin solution in the step S4 (1), a concentration of the EDTA is 0.01 wt % to 0.04 wt %; and a concentration of the trypsin is 0.25 wt % to 0.35 wt %.

Preferably, in the EDTA-containing trypsin solution in the step S4 (1), the concentration of the EDTA is 0.025 wt %; and the concentration of the trypsin is 0.3 wt %.

More specifically, the soaking in the EDTA-containing trypsin solution in step S4 (1) is performed at a temperature of 30° C. to 37° C., preferably 37° C., for 12 h to 24 h, preferably 24 h.

More specifically, soaking the pig skin in the detergent solution in the step S4 (1) is performed at a temperature of 24° C. to 32° C., preferably 30° C., for 12 h to 20 h, preferably 20 h.

More specifically, a concentration of the neutral protease solution in the step S4 (1) is 500 U/L to 700 U/L.

More specifically, soaking the pig skin in the neutral protease solution in the step S4 (1) is performed at a temperature of 28° C. to 35° C., preferably 35° C., for 12 h to 24 h, preferably 24 h.

More specifically, a concentration of the α-1,3-galactosidase solution in the step S4 (2) is 10 μg/mL to 20 μg/mL.

Preferably, soaking the non-immunogenic pig skin in the α-1,3-galactosidase solution in the step S4 (2) is performed at a temperature of 24° C. to 37° C., preferably 37° C., for 1 h to 4 h, preferably 3 h.

More specifically, the filter aid in the step S4 (2) is at least one selected from a group consisting of: diatomite, perlite, cellulose, asbestos, magnesium oxide, and activated carbon; and the diatomite is preferred.

More specifically, a mass ratio of the filter aid to the precipitate A in the step S4 (2) is (0.5-3):1.

More specifically, the cation exchange medium in the step S4 (2) is at least one selected from a group consisting of: DEAE(N,N diethylaminoethyl)Sepharose Fast Flow, Q(N,N-diethylamino-2-hydroxypropyl), and SP(sulfopropyl)-Sephadex; and the SP(sulfopropyl)-Sephadex is preferred.

More specifically, an elution solution for the elution and separation in the step S4 (2) is a 10 mM Tris-HCl aqueous solution containing 0.5 wt %-0.9 wt % of a sodium salt.

The modified polysaccharide in the step S4 (3) is a mixture of methacrylic anhydride-modified hyaluronic acid, oat β-glucan and aldehyde-modified hyaluronic acid at a mass ratio of 2:5:3.

A method for preparing the methacrylic anhydride (MA)-modified hyaluronic acid is as follows:
weighing and dissolving 1 g of a hyaluronic acid (HA) powder in 100 mL of deionized water and stirring well to obtain a HA solution with a concentration of 1%; then, adding 64 mL of MA dropwise slowly to the HA solution to react for 4 h, wherein a pH value of a reaction solution is adjusted and maintained at 8.0 during addition of MA, and the pH value of the reaction solution is maintained at 8.0 during reaction; and after the reaction is finished, washing the reaction solution with ethanol repeatedly to obtain a flocculent precipitate, performing centrifugation, and then re-dissolving a white precipitate obtained by centrifugation in deionized water, and performing dialysis on a resulting solution in a dialysis bag with a molecular weight cut off of 8,000-14,000 kDa for 5 days, and then drying a resulting product in a vacuum freeze dryer to obtain a modified sponge-like HAMA, namely the methacrylic anhydride (MA)-modified hyaluronic acid.

A method for preparing the aldehyde-modified hyaluronic acid is as follows:
dissolving hyaluronic acid (HA) with a molecular weight of 60 KDa and sodium periodate in deionized water separately to obtain a sodium periodate solution and a HA solution; and then, adding the sodium periodate solution dropwise slowly into the HA solution to react in dark at room temperature for 3 h; after reaction is finished, performing dialysis on a resulting solution in a dialysis bag with a molecular weight cut off of 8000-14000 kDa for 5 days, and then drying a resulting product in a vacuum freeze dryer to obtain the sponge-like aldehyde-modified hyaluronic acid.

The hyaluronic acid is moistuHA produced by Dongying First Biochem Industrial Co., Ltd., and a molecular weight of hyaluronic acid is between 20 KDa and 2000 KDa; the oat β-glucan (1%) is produced by Guangzhou Beta Health Biotechnology Co., Ltd., with a batch number of BT2212101005B.

In a second aspect, the present application provides a composition of triple-helix collagen and Antarctic glycoprotein prepared by the method described above.

In a third aspect, the present application provides a use of the composition of triple-helix collagen and Antarctic glycoprotein described above in a preparation of an anti-aging product; the anti-aging product includes a medical device, a medicine or a cosmetic.

In a fourth aspect, the present application provides an anti-aging cosmetic. The anti-aging cosmetic includes the composition of triple-helix collagen and Antarctic glycoprotein prepared by the method described above or the composition of triple-helix collagen and Antarctic glycoprotein described above.

Specifically, the anti-aging cosmetic includes but is not limited to a facial cleanser, a facial cleanser, a toner, a lotion, a facial cream, an essence, an eye cream, a facial mask, a foundation, a concealer, a sunscreen, a sunscreen spray, a shampoo, a hair conditioner, a body wash or a body lotion.

Specifically, the anti-aging cosmetic further includes a cosmetically acceptable additive.

Preferably, the cosmetically acceptable additive includes but is not limited to one or more of a diluent, an excipient, a filler, a binder, a wetting agent, a disintegrating agent, an emulsifier, a co-solvent, a solubilizer, an osmotic pressure regulator, a surfactant, a coating material, a colorant, a pH adjuster, an antioxidant, and a buffer agent.

In a fifth aspect, the present application provides an anti-aging medicine. The anti-aging medicine includes the composition of triple-helix collagen and Antarctic glycoprotein prepared by the method described above or the composition of triple-helix collagen and Antarctic glycoprotein described above.

Specifically, the anti-aging medicine further includes a pharmaceutically acceptable excipient.

Preferably, the pharmaceutically acceptable excipient includes, but is not limited to, a solvent, a diluent, a disintegrating agent, a precipitation inhibitor, a surfactant, a glidant, a binder, a lubricant, a dispersant, a suspending agent, an isotonic agent, a thickener, an emulsifier, a preservative, a stabilizer, a hydrating agent, an emulsification accelerator, a buffer agent, an absorbent, a colorant, a fragrance, a sweetener, an ion exchanger, a mold release agent, a coating agent, a corrigent or an antioxidant.

In a sixth aspect, the present application provides an anti-aging medical device. The anti-aging medical device includes the composition of triple-helix collagen and Antarctic glycoprotein prepared by the method described above or the composition of triple-helix collagen and Antarctic glycoprotein described above.

Specifically, the medical device includes medical equipment and a medical consumable.

Preferably, the medical device includes but is not limited to: a medical sanitary material and a dressing, a medical polymer material product, a medical metal material product, a medical optical instrument and apparatus, and endoscopic equipment.

The beneficial effects of the present application are as follows:

The composition of triple-helix collagen and Antarctic glycoprotein prepared by the method of the application has a protective effect on UVB induced Hacat cells, can effectively inhibit inflammatory cytokines and cellular oxidative stress levels, and is an anti-aging product.

### BRIEF DESCRIPTION OF THE DRAWINGS

FIG. 1 shows results of cell survival rate determination; in the figure, ** represents significant difference compared with a blank group, and p<0.01; *** represents significant difference compared with the blank group, and p<0.001; ###represents significant difference compared with a model group (p<0.001).

FIG. 2 shows results of inflammation and oxidative stress determination; in the figure, A represents an expression level of IFN-γ; B represents an expression level of IL-6; C represents an expression level of MDA; ###represents significant difference compared with a model group (p<0.001).

### DETAILED DESCRIPTION

The following non-limiting examples may enable those of ordinary skill in the art to more fully understand the present application, but do not limit the present application in any way. The following is only an exemplary description of the scope of the present application. Those skilled in the art can make various changes and modifications to the present application based on the disclosed content, and the changes and modifications should also fall within the scope of the present application.

The present application will be further explained in the specific embodiments below. The various chemical reagents used in the embodiments of the present application are obtained by conventional commercial channels unless otherwise specified. Unless otherwise specified, the operation steps is understood to be performed at room temperature.

The purchase information of all materials for the present application is shown in Table 1:

TABLE 1
Materials used

| | | Product |
|---|---|---|
| Description | Manufacturer | number |
| Cellulase | Shandong Longkete Enzyme | Enzyme activity |
| | Preparation Co., Ltd. | 100,000 U/g |
| Papain | Shandong Longkete Enzyme | Enzyme activity |
| | Preparation Co., Ltd. | 120,000 U/g |
| Neutral | Shandong Longkete Enzyme | Enzyme activity |
| protease | Preparation Co., Ltd. | 150,000 U/g |
| HaCaT cell | Shanghai Meilian | mlsw-3644 |
| | Biotechnology Co., Ltd. | |
| DMEM | Guangdong Huankai Microbial | XB01-01 |
| | Technology Co., Ltd. | |
| Hyaluronic | Dongying First Biochem | moistuHA |
| acid | Industrial Co., Ltd. | |
| Oat β-glucan | Guangzhou Beta Health | BT2212101005B |
| | Biotechnology Co., Ltd. | |
| Pseudoalteromonas | SHMCC | JCM18779 |
| 2216E liquid | Shanghai Yuanmu | YP01827 |
| medium | Biotechnology Co., Ltd. | |
| IFN-γ ELISA | Shenzhen Zike | ZK-F6259 |
| kit | Biotechnology Co., Ltd. | |
| IL-6 ELISA kit | Shenzhen Zike | ZK-F6240 |
| | Biotechnology Co., Ltd. | |
| MDA ELISA kit | Shenzhen Zike Biotechnology | ZK-F6255 |
| | Co., Ltd. | |

### Basic Experimental Example 1: Preparation of Methacrylic Anhydride (MA)-Modified Hyaluronic Acid

1 g of a hyaluronic acid (HA) powder was weighed and then dissolved in 100 mL of deionized water and stirred well to obtain a HA solution with a concentration of 100. Then, adding 64 mL of MA dropwise slowly to the HA solution to react for 4 h, wherein the pH value of a reaction solution is adjusted and maintained at 8.0 during addition of MA, and the pH of the reaction solution is maintained at 8.0 during the reaction. After the reaction was finished, the reaction solution was washed with ethanol repeatedly to obtain a flocculent precipitate and centrifugation was then performed to obtain a white precipitate, the white precipitate was then re-dissolved in deionized water, and a resulting solution was dialyzed in a dialysis bag with a molecular weight cut off of 8,000-14,000 kDa for 5 days, and then a dialysis retentate was dried in a vacuum freeze dryer to obtain a modified sponge-like HAMA, namely the methacrylic anhydride (MA)-modified hyaluronic acid.

### Basic Experimental Example 2: Preparation of Aldehyde-Modified Hyaluronic Acid

Hyaluronic acid (HA) with a molecular weight of 60 KDa and sodium periodate were dissolved in deionized water separately to obtain a sodium periodate solution and a HA solution. Then, the sodium periodate solution was added dropwise into the HA solution slowly to react in dark at room temperature for 3 h. After the reaction was finished, dialysis was performed on a resulting solution in a dialysis bag with a molecular weight cut off of 8000-14000 kDa for 5 days, and then a dialysis retentate was dried in a vacuum freeze dryer to obtain the sponge-like aldehyde-modified hyaluronic acid.

### Example 1: Preparation of Composition of Triple-Helix Collagen and Antarctic Glycoprotein

### 1. Preparation of Triple-Helix Collagen Hydrogel

A triple-helix collagen hydrogel was prepared according to the method described in patent CN115944545B. The specific method is as follows:

### (1) Pre-Treatment of Pig Skin:

A certain amount of animal pig skin was soaked in a 0.25 wt % trypsin solution containing 0.01 wt % EDTA at 30° C. for 24 h, and then rinsed; and then, the pig skin was soaked in a phosphate buffer solution (pH 6.0) containing 0.05 wt % of an ionic detergent at 24° C. for 24 h and shaken, and then rinsed to obtain pre-treated pig skin.

### (2) Non-Immunogenic Treatment of Pig Skin:

The pre-treated pig skin from the step (1) was soaked into a neutral protease solution with a concentration of 500 U/L at 28° C. for 24 h, and then rinsed; then, the pig skin was soaked in the phosphate buffer solution (pH 6.0) containing 0.05 wt % of the ionic detergent at 24° C. for 24 h and shaken, and then rinsed; steps (1) and (2) were repeated for 1-3 times to obtain non-immunogenic pig skin.

The non-immunogenic pig skin obtained after the treatments was soaked in a 10 μg/mL α-1,3-galactosidase solution at 24° C. for 4 h and then rinsed to obtain precipitate A.

### (3) Filter Aid Adsorption:

A diatomite solution was added to the precipitate A obtained in the step (2) and a resulting solution was stirred and filtered, and a supernatant was collected for later use.

### (4) Separation and Purification:

The supernatant collected in the step (3) was concentrated, then collagen in the supernatant was adsorbed by SP(sulfopropyl)-Sephadex, and then elution and separation were performed using a 10 mM Tris-HCl aqueous solution containing 0.5 wt % of a sodium salt to finally obtain an eluent.

### (5) Preparation of Hydrogel:

A modified polysaccharide solution with a concentration of 10 mg/mL was added to the eluent from the step (4) for modification to obtain a hydrogel, wherein a mass ratio of the eluent to the modified polysaccharide solution was 1:3.

### (6) Filtration and Sterilization:

Filtration and sterilization were performed on the hydrogel obtained in the step (5) with a 0.22 μm filter membrane to obtain a sterile soluble collagen hydrogel solution, namely the triple-helix collagen hydrogel.

The ionic detergent in the step (1) and step (2) was a mixture of sodium dodecyl sulfate and Triton X-200 at a mass ratio of 4:6.

A mass ratio of the filter aid to the precipitate A in the step (3) was 0.5:1.

The modified polysaccharide in the step (5) was a mixture of the methacrylic anhydride-modified hyaluronic acid obtained in basic Experimental Example 1, oat β-glucan and the aldehyde-modified hyaluronic acid obtained in basic Experimental Example 2 at a mass ratio of 2:5:3.

### 2. Preparation of MaAFP Antifreeze Protein Solution

### 1) Preparation of Medium:

Glutamic acid, proline, vitamin B1, and sucrose were added to a 2216E liquid medium to obtain a 2216E liquid medium containing the glutamic acid with a final concentration of 0.4 g/L, the proline with a final concentration of 0.3 g/L, the vitamin B1 with a final concentration of 20 mg/L, and the sucrose with a final concentration of 2 g/L.

### 2) Strain Culture:

Pseudoalteromonas was inoculated into the above medium at an inoculation amount of 5%, and cultured at 20° C., 200 r/min for 7 days to obtain a bacterial solution. The bacterial solution was centrifuged at 5000 rpm for 10 min, and a supernatant was collected and then filtered with a 0.22 μm filter membrane, and a first filtrate was collected.

### (3) Separation:

The first filtrate was then ultrafiltered at a pressure of 8 bar with an ultrafiltration membrane having a molecular cutoff of 30 kDa and a second filtrate was then collected. An ultrafiltered sample (the second filtrate) was then placed in a dialysis bag with a molecular cutoff of 10 kDa, and dialyzed at 25° C. for 24 h, using PBS (pH 7.4) as a dialysis medium. During the dialysis process, the buffer solution (PBS) was changed every 8 h. Then, a dialysis retentate was collected and lyophilized to obtain a powder.

### 4) Purification:

The powder was dissolved in PBS (pH 7.4) and a resulting solution was loaded on a Sephadex G-100 column. The elution with PBS (pH 7.4) was then performed, and an eluent was collected to obtain a MaAFP antifreeze protein solution.

### 3. Preparation of Composition of Triple-Helix Collagen and Antarctic Glycoprotein

### S1. Pre-Treatment of Antarctic Krill

Raw material, fresh Antarctic krill, was washed and then lyophilized in a vacuum freeze dryer. The lyophilized Antarctic krill was pulverized and then sieved with a 10-mesh sieve to obtain a uniform lyophilized Antarctic krill powder.

### S2. Enzymatic Hydrolysis:

The lyophilized Antarctic krill powder was added into distilled water at a liquid-to-material ratio of 1:50 g/mL to obtain a mixture, a pH value of the mixture was adjusted to 6.0-7.0, then an enzyme mixture was added at an amount being 0.75 wt % of a mass of the lyophilized Antarctic krill powder to enzymatically hydrolyze the lyophilized Antarctic krill powder at 55° C. for 3 h to obtain an enzymatic hydrolysate. The enzymatic hydrolysate was heated at 90° C. for 20 min and then centrifuged at 8000 rpm for 10 min, and a supernatant was then collected for later use.

The enzyme mixture consisted of a cellulase, papain and a neutral protease, and a weight ratio of the cellulase to the papain to the neutral protease was 2:1.5:1.

### S3. Dialysis:

The supernatant collected in the setp S2 was placed into a dialysis bag with a molecular cutoff of 10 kDa, then dialyzed using distilled water as a dialysis medium for 48 h, a resulting dialysate was concentrated to ⅕ of the original volume, and ethanol was then added with a final ethanol concentration being 75% (v/v). After standing overnight, a resulting solution was centrifuged at 8000 rpm for 10 min, and a precipitate was then collected and lyophilized to obtain a crude Antarctic krill glycoprotein product.

### S4. Refining:

The crude Antarctic krill glycoprotein product was dissolved in distilled water to obtain a crude Antarctic krill glycoprotein solution. The MaAFP antifreeze protein solution was added at a volume ratio of the MaAFP antifreeze protein solution to the crude Antarctic krill glycoprotein solution being 0.65:1 to obtain a mixture; proteins in the mixture was adsorbed using SP(sulfopropyl)-Sephadex, elution and separation were performed using a 10 mM Tris-HCl aqueous solution containing 0.5 wt % of a sodium salt, and an eluent was collected to obtain an Antarctic glycoprotein solution.

### S5. Mixing and Lyophilization:

The Antarctic glycoprotein solution and the triple-helix collagen hydrogel solution were mixed thoroughly at a volume ratio of 1.25:1 to obtain a mixture, and the mixture was then lyophilized to obtain the composition of triple-helix collagen and Antarctic glycoprotein.

### Example 2: Preparation of Composition of Triple-Helix Collagen and Antarctic Glycoprotein

### 1. Preparation of Triple-Helix Collagen Hydrogel

A triple-helix collagen hydrogel was prepared according to the method described in patent CN115944545B. The specific method is as follows:

### (1) Pre-Treatment of Pig Skin:

A certain amount of animal pig skin was soaked in a 0.35 wt % trypsin solution containing 0.04 wt % EDTA at 37° C. for 12 h, and then rinsed; and then, the pig skin was soaked in a phosphate buffer solution (pH 8.0) containing 0.05 wt % of an ionic detergent at 32° C. for 12 h and shaken, and then rinsed to obtain pre-treated pig skin.

### (2) Non-Immunogenic Treatment of Pig Skin:

The pre-treated pig skin from the step (1) was soaked into a neutral protease solution with a concentration of 600 U/L at 35° C. for 12 h, and then rinsed; then, the pig skin was soaked in the phosphate buffer solution (pH 8.0) containing 0.15 wt % of the ionic detergent at 32° C. for 12 h and shaken, and then rinsed; steps (1) and (2) were repeated for 1-3 times to obtain non-immunogenic pig skin.

The non-immunogenic pig skin obtained after the treatments was soaked in a 15 μg/mL α-1,3-galactosidase solution at 37° C. for 1 h and then rinsed to obtain precipitate A.

### (3) Filter Aid Adsorption:

A diatomite solution was added to the precipitate A obtained in the step (2) and a resulting solution was stirred and filtered, and a supernatant was collected for later use.

### (4) Separation and Purification:

The supernatant collected in the step (3) was concentrated, then collagen in the supernatant was adsorbed by SP(sulfopropyl)-Sephadex, and then elution and separation were performed using a 10 mM Tris-HCl aqueous solution containing 0.9 wt % of a sodium salt to finally obtain an eluent.

### (5) Preparation of Hydrogel:

A modified polysaccharide solution with a concentration of 40 mg/mL was added to the eluent from the step (4) for modification to obtain a hydrogel, wherein a mass ratio of the eluent to the modified polysaccharide solution was 5:1.

### (6) Filtration and Sterilization:

Filtration and sterilization were performed on the hydrogel obtained in the step (5) with a 0.22 μm filter membrane to obtain a sterile soluble collagen hydrogel solution, namely the triple-helix collagen hydrogel.

The ionic detergent in the step (1) and step (2) was a mixture of sodium dodecyl sulfate and Triton X-200 at a mass ratio of 4:6.

A mass ratio of the filter aid to the precipitate A in the step (3) was 3:1.

The modified polysaccharide in the step (5) was a mixture of the methacrylic anhydride-modified hyaluronic acid obtained in basic Experimental Example 1, oat β-glucan and the aldehyde-modified hyaluronic acid obtained in basic Experimental Example 2 at a mass ratio of 2:5:3.

### 2. Preparation of MaAFP Antifreeze Protein Solution

### 1) Preparation of Medium:

Glutamic acid, proline, vitamin B1, and sucrose were added to a 2216E liquid medium to obtain a 2216E liquid medium containing the glutamic acid with a final concentration of 0.2 g/L, the proline with a final concentration of 0.45 g/L, the vitamin B1 with a final concentration of 30 mg/L, and the sucrose with a final concentration of 3 g/L.

### 2) Strain Culture:

Pseudoalteromonas was inoculated into the above medium at an inoculation amount of 4%, and cultured at 15° C., 500 r/min for 5 days to obtain a bacterial solution. The bacterial solution was centrifuged at 8000 rpm for 10 min, and a supernatant was collected and then filtered with a 0.22 μm filter membrane, and a first filtrate was collected.

### (3) Separation:

The first filtrate was then ultrafiltered at a pressure of 5 bar with an ultrafiltration membrane having a molecular cutoff of 15 kDa and a second filtrate was then collected. An ultrafiltered sample (the second filtrate) was then placed in a dialysis bag with a molecular cutoff of 15 kDa, and dialyzed at 25° C. for 48 h, using PBS (pH 7.4) as a dialysis medium. During the dialysis process, the buffer solution (PBS) was changed every 8 h. Then, a dialysis retentate was collected and lyophilized to obtain a powder.

### 4) Purification:

The above powder was dissolved in PBS (pH 7.4) and a resulting solution was loaded on a Sephadex G-100 column. The elution with PBS (pH 7.4) was then performed, and an eluent was collected to obtain a MaAFP antifreeze protein solution.

### 3. Preparation of Composition of Triple-Helix Collagen and Antarctic Glycoprotein

### S1. Pre-Treatment of Antarctic Krill

Raw material, fresh Antarctic krill, was washed and then lyophilized in a vacuum freeze dryer. The lyophilized Antarctic krill was pulverized and then sieved with a 10-mesh sieve to obtain a uniform lyophilized Antarctic krill powder.

### S2. Enzymatic Hydrolysis:

The lyophilized Antarctic krill powder was added into distilled water at a liquid-to-material ratio of 1:40 g/mL to obtain a mixture, a pH value of the mixture was adjusted to 6.0-7.0, then an enzyme mixture was added at an amount being 0.75 wt % of a mass of the lyophilized Antarctic krill powder to enzymatically hydrolyze the the lyophilized Antarctic krill powder at 60° C. for 2 h to obtain an enzymatic hydrolysate. The enzymatic hydrolysate was heated at 100° C. for 15 min and then centrifuged at 5000 rpm for 20 min, and a supernatant was then collected for later use.

The enzyme mixture consisted of a cellulase, papain and a neutral protease, and a weight ratio of the cellulase to the papain to the neutral protease was 2:1.5:1.

### S3. Dialysis:

The supernatant collected in the setp S2 was placed into a dialysis bag with a molecular cutoff of 15 kDa, then dialyzed using distilled water as a dialysis medium for 48 h, a resulting dialysate was concentrated to ⅕ of the original volume, and ethanol was then added with a final ethanol concentration being 70% (v/v). After standing overnight, a resulting solution was centrifuged at 5000 rpm for 20 min, and a precipitate was then collected and lyophilized to obtain a crude Antarctic krill glycoprotein product.

### S4. Refining:

The crude Antarctic krill glycoprotein product was dissolved in distilled water to obtain a crude Antarctic krill glycoprotein solution. The MaAFP antifreeze protein solution was added at a volume ratio of the MaAFP antifreeze protein solution to the crude Antarctic krill glycoprotein solution being 0.3:1 to obtain a mixture; proteins in the mixture was adsorbed using SP(sulfopropyl)-Sephadex, elution and separation were performed using a 10 mM Tris-HCl aqueous solution containing 0.9 wt % of a sodium salt, and an eluent was collected to obtain an Antarctic glycoprotein solution.

### S5. Mixing and Lyophilization:

The Antarctic glycoprotein solution and the triple-helix collagen hydrogel solution were mixed thoroughly at a volume ratio of 0.4:1 to obtain a mixture, and the mixture was then lyophilized to obtain the composition of triple-helix collagen and Antarctic glycoprotein.

### Example 3: Preparation of Composition of Triple-Helix Collagen and Antarctic Glycoprotein

### 1. Preparation of Triple-Helix Collagen Hydrogel

A triple-helix collagen hydrogel was prepared according to the method described in patent CN115944545B. The specific method is as follows:

### (1) Pre-Treatment of Pig Skin:

A certain amount of animal pig skin was soaked in a 0.3 wt % trypsin solution containing 0.025 wt % EDTA at 37° C. for 24 h, and then rinsed; and then, the pig skin was soaked in a phosphate buffer solution (pH 7.2) containing 0.10 wt % of an ionic detergent at 30° C. for 20 h and shaken, and then rinsed to obtain pre-treated pig skin.

### (2) Non-Immunogenic Treatment of Pig Skin:

The pre-treated pig skin from the step (1) was soaked into a neutral protease solution with a concentration of 700 U/L at 35° C. for 24 h, and then rinsed; then, the pig skin was soaked in the phosphate buffer solution (pH 7.2) containing 0.10 wt % of the ionic detergent at 30° C. for 20 h and shaken, and then rinsed; steps (1) and (2) were repeated for 1-3 times to obtain non-immunogenic pig skin.

The non-immunogenic pig skin obtained after the treatments was soaked in a 20 μg/mL α-1,3-galactosidase solution at 37° C. for 3 h and then rinsed to obtain precipitate A.

### (3) Filter Aid Adsorption:

A diatomite solution was added to the precipitate A obtained in the step (2) and a resulting solution was stirred and filtered, and a supernatant was collected for later use.

### (4) Separation and Purification:

The supernatant collected in the step (3) was concentrated, then collagen in the supernatant was adsorbed by SP(sulfopropyl)-Sephadex, and then elution and separation were performed using a 10 mM Tris-HCl aqueous solution containing 0.8 wt % of a sodium salt to finally obtain an eluent.

### (5) Preparation of Hydrogel:

A modified polysaccharide solution with a concentration of 25 mg/mL was added to the eluent from the step (4) for modification to obtain a hydrogel, wherein a mass ratio of the eluent to the modified polysaccharide solution was 3.5:2.

### (6) Filtration and Sterilization:

Filtration and sterilization were performed on the hydrogel obtained in the step (5) with a 0.22 μm filter membrane to obtain a sterile soluble collagen hydrogel solution, namely the triple-helix collagen hydrogel.

The ionic detergent in the step (1) and step (2) was a mixture of sodium dodecyl sulfate and Triton X-200 at a mass ratio of 4:6.

A mass ratio of the filter aid to the precipitate A in the step (3) was 1:1.

The modified polysaccharide in the step (5) was a mixture of the methacrylic anhydride-modified hyaluronic acid obtained in basic Experimental Example 1, oat β-glucan and the aldehyde-modified hyaluronic acid obtained in basic Experimental Example 2 at a mass ratio of 2:5:3.

### 2. Preparation of MaAFP Antifreeze Protein Solution

### 1) Preparation of Medium:

Glutamic acid, proline, vitamin B1, and sucrose were added to a 2216E liquid medium to obtain a 2216E liquid medium containing the glutamic acid with a final concentration of 0.6 g/L, the proline with a final concentration of 0.15 g/L, the vitamin B1 with a final concentration of 15 mg/L, and the sucrose with a final concentration of 1 g/L.

### 2) Strain Culture:

Pseudoalteromonas was inoculated into the above medium at an inoculation amount of 3%, and cultured at 20° C., 200 r/min for 7 days to obtain a bacterial solution. The bacterial solution was centrifuged at 3000 rpm for 15 min, and a supernatant was collected and then filtered with a 0.22 μm filter membrane, and a first filtrate was collected.

### (3) Separation:

The firset filtrate was then ultrafiltered at a pressure of 8 bar with an ultrafiltration membrane having a molecular cutoff of 30 kDa and a second filtrate was then collected. An ultrafiltered sample (the second filtrate) was then placed in a dialysis bag with a molecular cutoff of 10 kDa, and dialyzed at 20° C. for 24 h, using PBS (pH 7.4) as a dialysis medium. During the dialysis process, the buffer solution (PBS) was changed every 8 h. Then, a dialysis retentate was collected and lyophilized to obtain a powder.

### 4) Purification:

The powder was dissolved in PBS (pH 7.4) and a resulting solution was loaded on a Sephadex G-100 column. The elution with PBS (pH 7.4) was then performed, and an eluent was collected to obtain a MaAFP antifreeze protein solution.

### 3. Preparation of Composition of Triple-Helix Collagen and Antarctic Glycoprotein

### S1. Pre-Treatment of Antarctic Krill

Raw material, fresh Antarctic krill, was washed and then lyophilized in a vacuum freeze dryer. The lyophilized Antarctic krill was pulverized and then sieved with a 10-mesh sieve to obtain a uniform lyophilized Antarctic krill powder.

### S2. Enzymatic Hydrolysis:

The lyophilized Antarctic krill powder was added into distilled water at a liquid-to-material ratio of 1:60 g/mL to obtain a mixture, a pH value of the mixture was adjusted to 6.0-7.0, then an enzyme mixture was added at an amount being 0.75 wt % of a mass of the lyophilized Antarctic krill powder to enzymatically hydrolyze the the lyophilized Antarctic krill powder at 50° C. for 4 h to obtain an enzymatic hydrolysate. The enzymatic hydrolysate was heated at 90° C. for 30 min and then centrifuged at 3000 rpm for 30 min, and a supernatant was then collected for later use.

The enzyme mixture consisted of a cellulase, papain and a neutral protease, and a weight ratio of the cellulase to the papain to the neutral protease was 2:1.5:1.

### S3. Dialysis:

The supernatant collected in the setp S2 was placed into a dialysis bag with a molecular cutoff of 14 kDa, then dialyzed using distilled water as a dialysis medium for 48 h, a resulting dialysate was concentrated to ⅕ of the original volume, and ethanol was then added with a final ethanol concentration being 80% (v/v). After standing overnight, a resulting solution was centrifuged at 3000 rpm for 30 min, and a precipitate was then collected and lyophilized to obtain a crude Antarctic krill glycoprotein product.

### S4. Refining:

The crude Antarctic krill glycoprotein product was dissolved in distilled water to obtain a crude Antarctic krill glycoprotein solution. The MaAFP antifreeze protein solution was added at a volume ratio of the MaAFP antifreeze protein solution to the crude Antarctic krill glycoprotein solution of 1:1 to obtain a mixture; proteins in the mixture was adsorbed using SP(sulfopropyl)-Sephadex, elution and separation were performed using a 10 mM Tris-HCl aqueous solution containing 0.75 wt % of a sodium salt, and an eluent was collected to obtain an Antarctic glycoprotein solution.

### S5. Mixing and Lyophilization:

The Antarctic glycoprotein solution and the triple-helix collagen hydrogel solution were mixed thoroughly at a volume ratio of 1.5:1 to obtain a mixture, and the mixture was then lyophilized to obtain the composition of triple-helix collagen and Antarctic glycoprotein.

### Comparative Example 1: Preparation of Composition of Triple-Helix Collagen and Antarctic Glycoprotein

Comparative Example 1 was designed with reference to Example 1. Comparative Example 1 only differed from Example 1 in the enzyme mixture in the enzymatic hydrolysis in the step S2.

The enzyme mixture in Comparative Example 1 consisted of a cellulase and papain, and a weight ratio of the cellulase to the papain was 2:2.5.

### Comparative Example 2: Preparation of Composition of Triple-Helix Collagen and Antarctic Glycoprotein

Comparative Example 2 was designed with reference to Example 1. Comparative Example 2 only differed from Example 1 in the enzyme mixture in the enzymatic hydrolysis in the step S2.

The enzyme mixture in Comparative Example 2 consisted of a cellulase and a neutral protease, and a weight ratio of the cellulase to the neutral protease was 2:2.5.

### Comparative Example 3: Preparation of Composition of Triple-Helix Collagen And Antarctic Glycoprotein

Comparative Example 3 was designed with reference to Example 3. Comparative Example 3 only differed from Example 1 in that: “an enzyme mixture” in the enzymatic hydrolysis in the step S2 was replaced with “a cellulase”.

### Comparative Example 4: Preparation of Composition of Triple-Helix Collagen and Antarctic Glycoprotein

Comparative Example 4 was designed with reference to Example 1. Comparative Example 4 only differed from Example 1 in the enzyme mixture in the enzymatic hydrolysis in the step S2.

The enzyme mixture in Comparative Example 4 consisted of a cellulase, papain and a neutral protease, and a weight ratio of the cellulase to the papain to the neutral protease was 1:1.5:2.

### Comparative Example 5: Preparation of Composition of Triple-Helix Collagen and Antarctic Glycoprotein

Comparative Example 5 was designed with reference to Example 1. Comparative Example 5 only differed from Example 1 in that: “an enzyme mixture was added at an amount being 0.75 wt % of a mass of the lyophilized Antarctic krill powder” in the enzymatic hydrolysis in the step S2 was replaced with “an enzyme mixture was added at an amount being 1.5 wt % of a mass of the lyophilized Antarctic krill powder”.

### Comparative Example 6: Preparation of Composition of Triple-Helix Collagen and Antarctic Glycoprotein

Comparative Example 6 was designed with reference to Example 1. Comparative Example 6 only differed from Example 1 in that: “2. Preparation of MaAFP antifreeze protein solution” was not included and “Step S4. Refining” was different.

### Step S4 In Comparative Example 6 was as Follows:

The crude Antarctic krill glycoprotein product was dissolved in distilled water to obtain a crude Antarctic krill glycoprotein solution. Physiological saline was added at a volume ratio of the physiological saline to the crude Antarctic krill glycoprotein solution being 0.65:1 to obtain a mixture; proteins in the mixture was adsorbed using SP(sulfopropyl)-Sephadex, elution and separation were performed using a 10 mM Tris-HCl aqueous solution containing 0.5 wt % of a sodium salt, and an eluent was collected to obtain an Antarctic glycoprotein solution.

### Comparative Example 7: Preparation of Lyophilized Antarctic Glycoprotein Powder

Comparative Example 7 was designed with reference to Example 1. Comparative Example 7 only differed from Example 1 in that: “1. Preparation of triple-helix collagen hydrogel” was not included and “Step S5: Mixing and lyophilization” was different.

Step S5 in Comparative Example 7 was as follows:

The Antarctic glycoprotein solution and physiological saline were mixed thoroughly at a volume ratio of 1.25:1 to obtain a mixture, and the mixture was then lyophilized to obtain the lyophilized Antarctic glycoprotein powder.

### Comparative Example 8: Preparation of Lyophilized Triple-Helix Collagen Hydrogel Powder

Comparative Example 8 was designed with reference to Example 1. Comparative Example 8 only differed from Example 1 in that: “2. Preparation of triple-helix collagen hydrogel” and “3. Preparation of composition of triple-helix collagen and Antarctic glycoprotein” were not included, and “Step S5: Mixing and lyophilization” was different.

Step S5 in Comparative Example 8 was as follows:

Physiological saline and the triple-helix collagen hydrogel solution were mixed thoroughly at a volume ratio of 1.25:1 to obtain a mixture, and the mixture was then lyophilized to obtain a lyophilized triple-helix collagen hydrogel powder.

### Comparative Example 9: Preparation of Composition of Triple-Helix Collagen and Antarctic Glycoprotein

Comparative Example 9 was designed with reference to Example 1. Comparative Example 9 only differed from Example 1 in “steps S4 and S5” in “3. Preparation of composition of triple-helix collagen and Antarctic glycoprotein”.

### S4. Refining:

The crude Antarctic krill glycoprotein product was dissolved in distilled water to obtain a crude Antarctic krill glycoprotein solution. The MaAFP antifreeze protein solution was added at a volume ratio of the MaAFP antifreeze protein solution to the crude Antarctic krill glycoprotein solution being 1.5:1 to obtain a mixture; proteins in the mixture was adsorbed using SP(sulfopropyl)-Sephadex, elution and separation were performed using a 10 mM Tris-HCl aqueous solution containing 0.5 wt % of a sodium salt, and an eluent was collected to obtain an Antarctic glycoprotein solution.

### S5. Mixing and Lyophilization:

The Antarctic glycoprotein solution and the triple-helix collagen hydrogel solution were mixed thoroughly at a volume ratio of 2:1 to obtain a mixture, and the mixture was then lyophilized to obtain the composition of triple-helix collagen and Antarctic glycoprotein.

### Experimental Example 1: Culture and Subculture of HaCaT Cells

The culture conditions of HaCaT cells: HaCaT cells were cultured in DMEM containing 10% FBS and 1% streptomycin solution at 37° C., 5% CO₂. When the cell density reached 80%-90%, subculture was performed. HaCaT cells were subcultured more than 2-3 times, and then cells in the logarithmic growth phase were taken for experiments.

### Experimental Example 2: Experiment on HaCaT Cell Survival Rate

1. The Experiment had 14 Groups, which were:
(1) Model control group reagent: DMEM containing 10% FBS and 1% streptomycin solution.
(2) Blank control group reagent: DMEM containing 10% FBS and 1% streptomycin solution.
(3) Reagent of Example 1 group: The composition of triple-helix collagen and Antarctic glycoprotein prepared in Example 1 was formulated with a DMEM containing 10% FBS and 1% streptomycin solution into a 2000 μg/mL solution.
(4) Reagent of Example 2 group: The composition of triple-helix collagen and Antarctic glycoprotein prepared in Example 2 was formulated with a DMEM containing 10% FBS and 1% streptomycin solution into a 800 μg/mL solution.
(5) Reagent of Example 3 group: The composition of triple-helix collagen and Antarctic glycoprotein prepared in Example 3 was formulated with a DMEM containing 10% FBS and 1% streptomycin solution into a 6000 μg/mL solution.
(6) Reagent of Comparative Example 1 group: The composition of triple-helix collagen and Antarctic glycoprotein prepared in Comparative Example 1 was formulated with a DMEM containing 10% FBS and 1% streptomycin solution into a 2000 μg/mL solution.
(7) Reagent of Comparative Example 2 group: The composition of triple-helix collagen and Antarctic glycoprotein prepared in Comparative Example 2 was formulated with a DMEM containing 10% FBS and 1% streptomycin solution into a 2000 μg/mL solution.
(8) Reagent of Comparative Example 3 group: The composition of triple-helix collagen and Antarctic glycoprotein prepared in Comparative Example 3 was formulated with a DMEM containing 10% FBS and 1% streptomycin solution into a 2000 μg/mL solution.
(9) Reagent of Comparative Example 4 group: The composition of triple-helix collagen and Antarctic glycoprotein prepared in Comparative Example 4 was formulated with a DMEM containing 10% FBS and 1% streptomycin solution into a 2000 μg/mL solution.
(10) Reagent of Comparative Example 5 group: The composition of triple-helix collagen and Antarctic glycoprotein prepared in Comparative Example 5 was formulated with a DMEM containing 10% FBS and 1% streptomycin solution into a 2000 μg/mL solution.
(11) Reagent of Comparative Example 6 group: The composition of triple-helix collagen and Antarctic glycoprotein prepared in Comparative Example 6 was formulated with a DMEM containing 10% FBS and 1% streptomycin solution into a 2000 μg/mL solution.
(12) Reagent of Comparative Example 7 group: The lyophilized Antarctic glycoprotein powder prepared in Comparative Example 7 was formulated with a DMEM containing 10% FBS and 1% streptomycin solution into a 2000 μg/mL solution.
(13) Reagent of Comparative Example 8 group: The lyophilized triple-helix collagen hydrogel powder prepared in Comparative Example 8 was formulated with a DMEM containing 10% FBS and 1% streptomycin solution into a 2000 μg/mL solution.
(14) Reagent of Comparative Example 9 group: The composition of triple-helix collagen and Antarctic glycoprotein prepared in Comparative Example 9 was formulated with a DMEM containing 10% FBS and 1% streptomycin solution into a 2000 μg/mL solution.

### 2. Cell Survival Rate Determination:

HaCaT cells in the logarithmic growth phase were prepared into cell suspension, and seeded into a 96-well plate at a density of 1×10⁴ cells per well (100 μL/well). After incubation at 37° C. in a 5% CO₂ incubator for 24 h, the supernatant was discarded. 100 μL of reagents were added according to experimental groups, followed by two hours of incubation. The experimental groups other than the blank control group were subjected to UVB irradiation at 300 nJ/cm² for 15 min. After further culture for 24 h, 100 μL of a basic medium containing 10% CCK-8 was added, the cells were incubated in a carbon dioxide incubator for 2 h, and the absorbance was measured with a microplate reader at 450 nm to calculate cell survival rate.

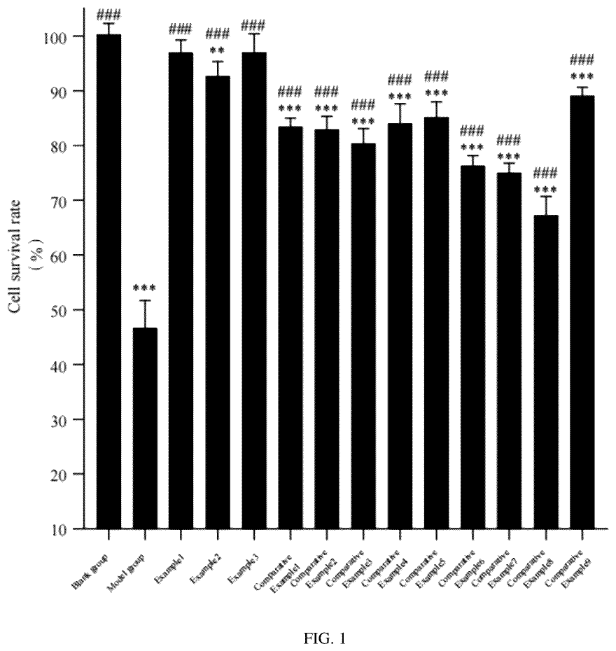

The results of the determination are shown in FIG. 1. Compared with the normal group, the model group showed a decrease of the cell survival rate to 46.75%±5.00%, proving that the cell model was successful, and the Hacat cells in this case showed photoaging. After intervention with the composition of triple-helix collagen and Antarctic glycoprotein prepared in Comparative Example 6, the lyophilized Antarctic glycoprotein powder prepared in Comparative Example 7, and the lyophilized Antarctic glycoprotein powder prepared in Comparative Example 8, the cell survival rate increased to 67.47%-76.28%, showing certain anti-aging effect. Examples 1-3 showed the best anti-aging effect and were able to maintain the survival rate of Hacat cells to 92.78%-97.18%, which was basically consistent with the effect of blank cells. Therefore, the compositions of triple-helix collagen and Antarctic glycoprotein prepared in Examples 1-3 were selected for subsequent inflammation and oxidative stress determination.

### Experimental Example 3: Inflammation and Oxidative Stress Determination

1. The Experiment had 5 Groups, which were:
(1) Model control group reagent: DMEM containing 10% FBS and 1% streptomycin solution.
(2) Blank control group reagent: DMEM containing 10% FBS and 1% streptomycin solution.
(3) Reagent of Example 1 group: The composition of triple-helix collagen and Antarctic glycoprotein prepared in Example 1 was formulated with a DMEM containing 10% FBS and 1% streptomycin solution into a 2000 μg/mL solution.
(4) Reagent of Example 2 group: The composition of triple-helix collagen and Antarctic glycoprotein prepared in Example 2 was formulated with a DMEM containing 10% FBS and 1% streptomycin solution into a 800 μg/mL solution.
(5) Reagent of Example 3 group: The composition of triple-helix collagen and Antarctic glycoprotein prepared in Example 3 was formulated with a DMEM containing 10% FBS and 1% streptomycin solution into a 6000 μg/mL solution.

### 2. Inflammation and Oxidative Stress Determination

HaCaT cells in the logarithmic growth phase were prepared into cell suspension, and seeded into a 96-well plate at a density of 6×10⁵ cells per well (100 μL/well). After incubation at 7° C. in a 5% CO₂ incubator for 24 h, the supernatant was discarded. 100 μL of reagents were added according to experimental groups, followed by two hours of incubation. The experimental groups other than the blank control group were subjected to UVB irradiation at 300 nJ/cm² for 15 min. After further culture for 24 h, the supernatant was collected by centrifugation, and the contents of IFN-7, IL-6, and MDA were determined according to the instructions of an ELISA kit.

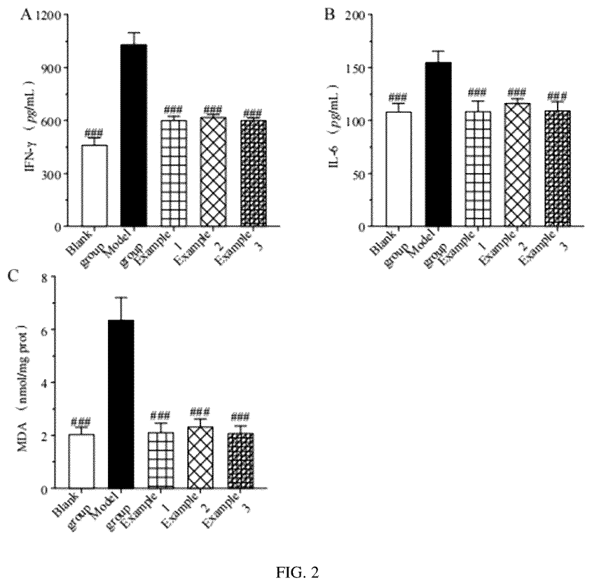

The results of the determination are shown in FIG. 2. The compositions prepared in Examples 1-3 of the present application can effectively inhibit the expression levels of inflammatory cytokines of IFN-7 and IL-6 and the production of MDA.

The above detailed description is a specific description of a possible embodiment of the application, and the embodiment is not intended to limit the scope of the application. It should be pointed out that all equivalent implementations or modifications that do not depart from the application should be included within the scope of the technical solutions of the application. Therefore, the scope of the application shall be subject to the appended claims.

What is claimed is:

1. A method for preparing a composition of triple-helix collagen and Antarctic glycoprotein, comprising:
step S1, adding a lyophilized Antarctic krill powder to distilled water to obtain a first mixture, adjusting a pH value of the first mixture, then adding an enzyme mixture to the first mixture for enzymatic hydrolysis, and then performing inactivation and collecting a first supernatant;
step S2, performing dialysis and concentration on the first supernatant, and then adding ethanol, standing still overnight, collecting a precipitate, and lyophilizing the precipitate to obtain a crude Antarctic krill glycoprotein product;
step S3, dissolving the crude Antarctic krill glycoprotein product in distilled water to obtain a crude Antarctic krill glycoprotein solution, adding an MaAFP antifreeze protein solution into the crude Antarctic krill glycoprotein solution to obtain a second mixture, using a cation exchange medium to adsorb proteins in the second mixture, and then performing elution and separation, and collecting a first eluent which is an Antarctic glycoprotein solution; and
step S4, mixing the Antarctic glycoprotein solution and a triple-helix collagen hydrogel solution at a volume ratio of (0.4-1.5):1 to obtain a third mixture, and lyophilizing the third mixture to obtain the composition of triple-helix collagen and Antarctic glycoprotein; wherein:
the enzyme mixture in the step S1 consists of a cellulase, papain and a neutral protease, and a weight ratio of the cellulase to the papain to the neutral protease is 2:1.5:1;
an amount of the enzyme mixture added in the step S1 is 0.5 wt % to 1.0 wt % of a mass of the lyophilized Antarctic krill powder;
a volume ratio of the MaAFP antifreeze protein solution to the crude Antarctic krill glycoprotein solution in the step S3 is (0.3-1.0):1;
an amino acid sequence of the MaAFP antifreeze protein in the step S3 is as shown in SEQ ID NO 1; and
a method for preparing the triple-helix collagen hydrogel solution in the step S4 comprises:
step (1) soaking pig skin in an ethylene diamine tetraacetic acid (EDTA)-containing trypsin solution, and then rinsing; then, soaking the pig skin in a detergent solution and shaking, and then rinsing; then, soaking the pig skin in a neutral protease solution, and then rinsing; and then soaking the pig skin in the detergent solution and shaking, and then rinsing; and repeating the above steps for 1-3 times to obtain non-immunogenic pig skin;
step (2) soaking the non-immunogenic pig skin in an α-1,3-galactosidase solution, and then rinsing to obtain precipitate A; adding a filter aid solution to the precipitate A, then stirring, filtering, and collecting a second supernatant; concentrating the second supernatant, then using a cation exchange medium to adsorb collagen in the second supernatant, and then performing elution and separation to finally obtain a second eluent;
step (3) adding a modified polysaccharide solution with a concentration of 10 mg/mL to 40 mg/mL to the second eluent from the step (2) for modification to obtain a hydrogel, and a mass ratio of the second eluent to the modified polysaccharide solution is 5:1-1:3; and
step (4) performing filtration and sterilization on the hydrogel obtained in the step (3) with a 0.22 μm filter membrane to obtain the triple-helix collagen hydrogel solution;
the detergent solution in the step (1) is a sodium phosphate buffer with a pH value of 6.0-8.0, containing 0.05%-0.15 wt % of an ionic detergent; and the ionic detergent is a mixture of sodium dodecyl sulfate and p-Octylphenoxyethoxyethyl sodium sulfonate at a mass ratio of 4:6;
the modified polysaccharide in the step (3) is a mixture of methacrylic anhydride-modified hyaluronic acid, oat β-glucan and aldehyde-modified hyaluronic acid at a mass ratio of 2:5:3.

2. The method according to claim 1, wherein at least one of:
a ratio of the lyophilized Antarctic krill powder to the distilled water in the step S1 is 1:(40-60) g/mL;
the pH value in the step S1 is 6.0 to 7.0; or
the enzymatic hydrolysis in the step S1 is performed at a temperature of 50° C. to 60° C. for 2 h to 4 h.

3. The method according to claim 1, wherein a method for preparing the MaAFP antifreeze protein solution in the step S3 comprises:
step (a) inoculating pseudoalteromonas into a medium containing glutamic acid, proline, vitamin B1 and sucrose at an inoculation amount of 3% to 5%, and performing culture to obtain a bacterial solution;
step (b) performing centrifugation on the bacterial solution and then collecting a third supernatant, filtering the third supernatant, and collecting a filtrate;
step (c) performing dialysis on the filtrate, and collecting and lyophilizing a dialysis retentate to obtain a powder; and
step (d) dissolving the powder to obtain a mixture, using a cation exchange medium to adsorb proteins in the mixture and then performing elution and separation, and collecting a third eluent to obtain the MaAFP antifreeze protein solution.

4. The method according to claim 3, wherein the medium in the step (a) is a medium containing the glutamic acid with a final concentration of 0.2 g/L to 0.6 g/L, the proline with a final concentration of 0.15 g/L to 0.45 g/L, the vitamin B1 with a final concentration of 15 mg/L to 30 mg/L, and the sucrose with a final concentration of 1 g/L to 3 g/L.

5. The method according to claim 1, wherein a solution used for the elution in the step S3 is a 10 mM Tris-HCl aqueous solution containing 0.5 wt %-0.9 wt % of a sodium salt.

6. A composition of triple-helix collagen and Antarctic glycoprotein prepared by the method according to claim 1.

7. Use of the composition of triple-helix collagen and Antarctic glycoprotein according to claim 6 in a preparation of an anti-aging product, wherein the anti-aging product comprises a dressing, a medicine or a cosmetic.

8. An anti-aging cosmetic, wherein the anti-aging cosmetic comprises the composition of triple-helix collagen and Antarctic glycoprotein according to claim 6.

9. An anti-aging medicine, wherein the anti-aging medicine comprises the composition of triple-helix collagen and Antarctic glycoprotein according to claim 6.


SEQUENCE LISTING (1 sequence)

SEQ ID NO: 1 - protein, 217 aa, synthetic construct
GSGNDQIVIDNDVEKWLDAGDGDNSIYVGDDVNRNNSAGIKTGSGDDDIFVKDNVDSTIQTGGGNDRVQIGYDLGNGYHSAHINLGDGDNNLIIKNDVNNYSTVHSGSGDDVVSIGDDVRYDADIQLGDGNDRLTIGDKIEKEVSINLGSGNDVLVVGGKVSDEAWVDGGSGSDSVWFESYSRSDYNSDKDGIKSRFANFENFKFSDGTVIGNASAF